(19) United States
(12) Patent Application Publication
(10) Pub. No.: US 2016/0341710 A1
(43) Pub. Date: Nov. 24, 2016

(54) A METHOD FOR DETERMINATION OF A QUALITY PARAMETER OF A HYDROCARBON GAS MIXTURE

(71) Applicant: NANONORD A/S, Alborg (DK)

(72) Inventor: Ole Norgaard Jensen, Alborg (DK)

(21) Appl. No.: 15/104,594

(22) PCT Filed: Dec. 10, 2014
(86) PCT No.: PCT/DK2014/050424

§ 371 Date: Jun. 15, 2016

(30) Foreign Application Priority Data
Dec. 19, 2013 (DK) PA 2013 70791

(51) Int. Cl. G01N 33/22; G01N 24/08

(52) U.S. Cl. CPC G01N 33/225; G01N 24/08

(57) ABSTRACT

The invention relates to a method for determination of a quality parameter of a hydrocarbon gas mixture, such as LNG, CNG or SNG, the method comprising subjecting at least a part of the hydrocarbon gas mixture to an NMR reading comprising generating a ¹H data comprising a ¹H NMR spectra and correlating the ¹H NMR data to calibration data, wherein the calibration data relates the ¹H NMR data to at least one quality parameter of the hydrocarbon gas. The invention also relates to a system suitable for determination of a quality parameter of a hydrocarbon gas mixture according to the method.

### TECHNICAL FIELD

The invention relates to a method and a system for determination of a quality parameter of a hydrocarbon gas mixture, such as a liquefied natural gas, a compressed natural gas or a synthetic natural gas.

### BACKGROUND ART

Hydrocarbon gas mixtures are important energy resources and the amount of recovered hydrocarbon gas mixture is constantly increasing.

Hydrocarbon gas mixtures such as natural gas consist primarily of methane, but may include varying amounts of other higher alkanes and even a lesser percentage of carbon dioxide, nitrogen, and hydrogen sulfide. Natural gas is an energy source often used for heating, cooking, and electricity generation. It is also used as fuel for vehicles and as a chemical feedstock in the manufacture of plastics and other commercially important organic chemicals. Natural gas is a hydro carbon gas formed when layers of buried plants and animals are exposed to intense heat and pressure over thousands of years. The energy that the plants originally obtained from the sun is stored in the form of carbon in natural gas. Natural gas can also be synthetized. Before natural gas can be used as a fuel, it must often undergo processing to remove impurities, including water, to meet the specifications of marketable natural gas.

The term “hydrocarbon gas mixture” is used herein to include liquefied natural gas (LNG), compressed natural gas (CNG) as well as synthetic natural gas (SNG).

LNG is natural gas that has been converted to a liquid form for ease of storage or transport. Liquefied natural gas takes up about 1/600th the volume of natural gas in the gaseous state at atmospheric condition. It is substantially odorless, colorless, non-toxic and non-corrosive. Methods of liquefying the natural gas are well known. The liquefaction process often involves removal of certain components, such as dust, acid gases, helium, water, and heavy hydrocarbons. The natural gas is then condensed into a liquid e.g. close to atmospheric pressure by cooling it to approximately −162° C.

LNG achieves a higher reduction in volume than compressed natural gas (CNG) so that the volumetric energy density of LNG is 2.4 times greater than that of CNG. This makes LNG cost efficient to transport over long distances. For many years LNG has primarily been used for transporting natural gas to markets, where it is regasified and distributed as pipeline natural gas. In more and more applications the LNG is directly used with or without regasification before usage. In the coming years it is expected that the LNG will for example commonly be used directly as fuel in vessels, trucks, and power plants. This leads to increased requirements for reliable means of measuring quality parameters of the LNG all the way to the consumer.

Due to the differences in composition from one hydrocarbon gas mixture to another the energy content in the hydrocarbon gas mixture varies. Further the hydrocarbon gas mixture may be more or less inhomogeneous which may also lead to variations in quality such as calorific quality e.g. energy content and combustion quality. Where the gas is to be consumed in an engine the knock resistance of the gas is an important quality factor. Engine knock can be very damaging to an engine and is usually caused by fuel self-ignition of low-grade fuels, although other factors can also lead to knock. The knock resistance of a hydrocarbon gas mixture mainly depends on the mixture of the gas—see e.g. U.S. Pat. No. 6,061,637.

In the past, measurement of the compositional properties of natural was typically accomplished by extracting a sample of the natural gas and then using gas chromatography to determine its constituent parts and from this calculating a calorific value. This sampling and analysis process is difficult, costly and rather hazardous in particular where the sampled streams included mixtures of methane, ethane, and propane at very low temperatures and/or high pressure. Further this method is time demanding and the tested samples cannot be reused.

GB 2349216 describes the use of thermal conductivity to determine the ratio of different compounds in LNG. However, this method is very time consuming and results in highly inaccurate determinations.

U.S. Pat. No. 7,213,413 describes the use of Raman spectrometers in a hydrocarbon processing plant for online measurement of at least one compositional property of a hydrocarbon stream. The Raman spectrometers are especially advantageous when employed to determine heavy hydrocarbons in a liquefied natural gas (LNG) plant. However, the Raman equipment is very expensive and not very suitable for determining the amount of the lower hydrocarbon compounds, such as methane, ethane, propane and butane.

The object of the invention is to provide a method and system for determination of a quality parameter of a hydrocarbon gas mixture which method and system is non-destructive, very reliable and simple to use.

### DISCLOSURE OF INVENTION

This object has been solved by the present invention as defined in the claims. The method or the system of the invention has shown to have a large number of advantages which will be clear from the following description.

The present invention provides a highly improved and non-destructive method which is very reliable and fast, can measure on large amounts of hydrocarbon gas mixture which in practice means that the method of the invention is also applicable on in-homogeneous hydrocarbon gas mixtures. Further, the method of the invention can be used in a very flexible way e.g. by performing the measurements on site and/or in-line or by taking suitable samples.

It should be emphasized that the term “comprises/comprising” when used herein is to be interpreted as an open term, i.e. it should be taken to specify the presence of specifically stated feature(s), such as element(s), unit(s), integer(s), step(s) compound(s) and combination(s) thereof, but does not preclude the presence or addition of one or more other stated features.

Reference made to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the skilled person will understand that particular features, structures, or characteristics may be combined in any suitable manner within the scope of the invention as defined by the claims.

The term “substantially” should herein be taken to mean that ordinary product variances and tolerances are comprised.

Although through many years there has been a need for a fast and effective method of determining the quality of a hydrocarbon gas mixture and in particular of the LNG type it has heretofore never been considered using NMR reading in such determinations. The inventor of the present invention has provided a method for determination of a quality parameter of a hydrocarbon gas mixture which is surprisingly reliable and simple in use.

The method of the invention comprises subjecting at least a part of the hydrocarbon gas mixture to an NMR reading comprising generating a ¹H data comprising a ¹H NMR spectra and correlating the ¹H NMR data to calibration data, wherein the calibration data relates the ¹H NMR data to at least one quality parameter of the hydrocarbon gas.

NMR Spectrometers are well known in the art and the skilled person will be able to select a suitable spectrometer for use in the present invention based on the teaching provided herein. Examples of spectrometer are e.g. described in U.S. Pat. No. 6,310,480 and in U.S. Pat. No. 5,023,551.

A NMR spectrometer comprises a unit for providing a permanent field e.g. a permanent magnet assembly, as well as a transmitter and a receiver for transmitting and/or receiving RF frequency pulses/signals. The RF receiver and RF transmitter are connected to an antenna or an array of RF antennas. The medium being measured is simultaneously exposed to the permanent magnet field and the RF antenna radio pulses. The antennas may be in the form of transceivers capable of both transmitting and receiving. The spectrometer further comprises at least one computing element, in the following referred to as a computer.

General background of NMR formation evaluation can be found e.g. in U.S. Pat. No. 5,023,551 and in WO 2013/087076. A general background description of NMR reading can further be found in “NMR Logging Principles and Applications” by George R. Coates et al, Halliburton Energy Services, 1999.

Although ‘NMR reading’ in the following often will be used in singular to describe the invention, it should be observed that the singular term ‘NMR reading’ also includes a plurality of NMR readings unless otherwise specified.

The terms ‘NMR reading’ and ‘NMR Measurement’ are used interchangeably.

The term “quality parameter” means any parameter that can be correlated to a quality of the hydrocarbon gas mixture. The quality parameter is preferably a calorific parameter and/or combustions quality parameter and/or a mass parameter.

A calorific parameter is a parameter that can be correlated to the energy content in the hydrocarbon gas mixture. The calorific parameter can for example correlate to a relative energy content or to an exact energy content of an amount of the hydrocarbon gas mixture e.g. of a mass amount or a volume amount at a specified condition (temperature and pressure) or of a specified batch of the hydrocarbon gas mixture.

A combustion quality parameter means a parameter that correlates to the combustion quality of the hydrocarbon gas mixture, such as knock resistance.

A mass parameter is a parameter that correlates to the mass of one or more compounds of the gas, such as of the entire hydrocarbon gas mixture.

Examples that influence the quality parameters include a quantitative and/or a relative value of methane, ethane, propane and/or butane, ═CH1- moiety, —CH2- moiety —CH3 moiety and/or CH4 moiety and/or any combinations thereof optionally in combination with any other parameter such as the content of inert compounds such as nitrogen and/or carbon dioxide.

The term moiety is herein used to designate a part or all of a molecule, such as specific groups of atoms or atomic bonds within a molecule that may include either whole functional groups or parts of functional groups as substructures.

In principle the quality parameter can be any parameter that provides an indication of the quality of the hydrocarbon gas mixture. Preferably the quality parameter can be any parameter that provides an indication of the quality of the hydrocarbon gas mixture with respect to its energy content and/or with respect to its combustions quality.

The phrase “energy content” means energy of the hydrocarbon gas mixture or of a compound of the hydrocarbon gas mixture e.g. the energy content of methane of the hydrocarbon gas mixture. The energy content can be in the form of energy concentration, energy per unit volume or mass, in form of a relative energy parameter or in form of derivatives thereof such as energy per hour of a flowing hydrocarbon gas mixture.

Natural gas energy content is typically measured in Gigajoules (GJ) or in the British thermal unit (BTU). A BTU is equal to about 1055 joules.

The term “combustion quality” is herein used to mean the quality of the hydrocarbon gas mixture with respect to one or more combustion properties of the mixture. A particularly preferred combustion quality parameter of the hydrocarbon gas mixture is a parameter indication or specifying the knock resistance of a hydrocarbon gas mixture. As mentioned above the knock resistance of a hydrocarbon gas mainly depends on the mixture of the gas. It has been found that the knock resistance decreases as the average hydrocarbon chain length increases. It has also been found that inert gas compounds of hydrocarbon gas mixture may add to an increase in knock resistance quality. Further information about knock resistance and prediction of the knock resistance based on the composition of the hydrocarbon gas mixture can e.g. be found in “Knock prediction in gas-fired reciprocating engines” by A. Trijselaar, University of Twente, January 2012.

The calibration data is advantageously based on calculations or determinations of a plurality of different hydrocarbon gas mixtures which are preferably known or at least comprise at least one known parameter, such as a content of at least one compound of the hydrocarbon gas mixture. The calibration date is preferably arranged to form a calibration map. Further information about the calibration data and how to obtain the calibration data is provided below.

To achieve an even more reliable determination of at least one quality parameter of the hydrocarbon gas mixture the method advantageously comprises subjecting at least a part of the hydrocarbon gas mixture to an NMR reading comprising generating a ¹³C data comprising a ¹³C NMR spectra and correlating the ¹³C NMR data to calibration data, wherein the calibration data relates the ¹³C NMR data to at least one quality parameter of the hydrocarbon gas.

By performing a ¹³C NMR reading on the hydrocarbon gas mixture a particularly reliable quality parameter can be obtained and in particular a calorific parameter of high reliability can be obtained.

Further additional compounds may be determined using NMR reading. Advantageously the method comprising subjecting at least a part of the hydrocarbon gas mixture to an NMR reading comprising generating a ¹⁴N data comprising a ¹⁴N NMR spectra and correlating the ¹⁴N NMR data to calibration data, wherein the calibration data relates the ¹⁴N NMR data to at least one quality parameter of the hydrocarbon gas. As mentioned above, the content of nitrogen and other inert gasses usually will have a high impact on the knock resistance of the hydrocarbon gas. In an embodiment the amount of carbon dioxide is determined based on the ¹³C NMR reading.

In an embodiment the method comprises determination of a content of nitrogen atoms; preferably the method comprises determining the concentration of nitrogen atoms and/or an amount of nitrogen atoms relative to an amount of carbon atoms and/or hydrogen atoms. The relative amount of content of nitrogen atoms can advantageously be applied to provide a quality parameter relating to the energy and/or mass content.

The phrase “content of a compound” means concentration of the compound, specific amount of the compound and/or relative amount of the compound in question with respect to one or more other compounds in the hydrocarbon gas mixture.

The hydrocarbon gas mixture can in principle be any kind of hydrocarbon gas mixture. The hydrocarbon gas mixture is advantageously a natural gas in the form of a mixture of several hydrocarbon gases, including methane (typically between 70% and 95%), ethane, propane, butane and pentane, as well as optionally carbon dioxide, nitrogen and hydrogen sulphide. Preferably the hydrocarbon gas mixture is substantially free of hydrogen sulphide, but where hydrogen sulphide is present it may be advantageous to perform a NMR reading on the ³³S isotope. A ³³S NMR reading preferably together with a 1H NMR reading e.g. in the form of cross polarization reading(s), preferably in the form of DEPT (Distortionless Enhancement by Polarization Transfer) can provide a quality parameter shoving a possible content of hydrogen sulphide.

The hydrocarbon gas mixture is preferably LNG, CNG or SNG or a mixture thereof. Most preferably the hydrocarbon gas mixture is LNG.

A mentioned above LNG is produced when natural gas is cooled to a very low temperature. During the liquefaction the natural gas is advantageously cooled below its boiling point, whereby certain concentrations of hydrocarbons, water, carbon dioxide, oxygen, and some sulfur compounds are either reduced or removed. The pressure of the LNG is usually below 2 bars such as about atmospheric pressure.

In the embodiment where the hydrocarbon gas mixture is LNG the NMR reading or readings are advantageously performed at a temperature below about −100° C., such as below about −150° C. and more preferably about −162° C.

In an embodiment the method comprises quantitative determination of one or more hydrogen containing compounds. Advantageously the method comprises quantitative determination of one or more hydrogen containing compounds selected from methane, ethane, propane, butane and any combinations thereof. Specific amounts of higher order hydrogen containing compounds and/or even cyclic hydrogen containing compounds may also be determined but in most situations this will not be required. Since the amount of such higher order hydrogen containing compounds and/or cyclic hydrogen containing compounds usually is relatively low.

Preferably the method comprises determining the concentration of hydrogen atoms in the hydrocarbon gas mixture. In particular where the hydrocarbon gas mixture is LNG the amount of non-hydrocarbon H containing compounds will be very low and therefore the concentration of hydrogen atoms in the LNG can be directly correlated to a level of energy content.

In an embodiment the method comprises quantitative determination of one or more carbon containing compounds, preferably selected from methane, ethane, propane, butane and any combinations thereof, the method preferably comprises determining the total concentration of carbon atoms in the hydrocarbon gas mixture.

In an embodiment the method comprises determination of at least one relative or specific concentration of methane, ethane, propane, n-butane, iso-butane, n-pentane, iso-Pentane, n-hexane and/or any combinations thereof in the hydrocarbon gas mixture.

The specific or relative amount of one or more of the hydrocarbon compounds in the hydrocarbon gas mixture can be applied to calculate the energy content and or to provide any other suitable quality parameter.

In an embodiment of the method of the invention the at least one quality parameter is selected from the content of at least one moiety comprising a H—C bond, preferably the at least one quality parameter is selected from the content of one or more of the moieties ═CH₁— moiety, —CH₂— moiety, —CH₃ moiety and/or CH₄ moiety and/or any combinations thereof. By determining the content of one or more moieties comprising an H—C bond the energy content can be directly estimated or even calculated e.g. based on calibration data and/or based on stoichiometric combustion estimations.

In an embodiment the at least one quality parameter is selected from the content of at least one hydrocarbon compound, preferably the at least one quality parameter is selected from the content of one or more of the compounds methane, ethane, propane, butane and any combinations thereof

In an embodiment the quality parameter comprises a calorific parameter, such as an energy content value, preferably in the form of the energy concentration per mass unit or per volume unit at a specified pressure and temperature of the hydrocarbon gas mixture.

When the hydrocarbon gas mixture to be read is provided in a gaseous state it is required to also know the temperature and pressure. Since the temperature may influence the NMR readings it is in general desirable to take this into account when preparing the calibration data and when performing the NMR readings. Preferably also the pressure is taken into account when preparing the calibration data and when performing the NMR readings in order to increase the accuracy of the determination of the one or more quality parameters.

In an embodiment the energy content value is the total energy content of the hydrocarbon gas mixture e.g. of a mass or volume unit of the gas mixture.

In an embodiment the energy content value is the total energy content of a plurality of the hydrocarbon compounds including at least methane.

The determination of the energy content is advantageously based on the content of C atoms and H atoms. It has been found that a surprisingly accurate determination of energy content can be provided based on the relative amount of C atoms and H atoms preferably combined with at least one of a specific content (e.g. the concentration) of at least one of C atoms and H atoms and/or a relative or specific content of N atoms.

In an embodiment the determination is based on the assumption that the major amount of hydrocarbon compounds is in the form of methane. Based on an assumption that the remaining hydrocarbons are ethane or C2-Cx hydrocarbons where x is from 3-7 the energy content can be determined with an acceptable accuracy.

In an embodiment the method comprises determining the energy content parameter based on reaction stoichiometry of a reaction equation of one or more of the respective hydrocarbon compounds in the hydrocarbon gas mixture.

Preferably the method comprises determining the energy content parameter based on reaction stoichiometry of the reaction equation of at least the reaction of methane to carbon dioxide and water.

The reaction equation applied may advantageously be CₙHₘ+(n+m/4)O₂->nCO₂+m/2H₂O, where n is an integer from 1 to 10 and m is 2n+2.

For even higher accuracy the method may comprise determining the energy content parameter based on reaction stoichiometry of the reaction equation of a plurality, such as of all of the respective hydrocarbon compounds e.g. up to C10 hydrocarbon, preferably up to C6 hydrocarbon in the hydrocarbon gas using the reaction equation CₙHₘ+(n+m/4)O₂->nCO₂+m/2H₂O, where n is an integer from 1 to 10, preferably from 1 to 6 and where m is 2n+2.

In an embodiment of the invention it has been found that the determination of the energy content advantageously can be based on Hess law on enthalpy of at least one reaction equation, such as at least one reaction equation where a hydrocarbon compound is reacted with oxygen to form carbon dioxide and water.

In order to provide an embodiment with a very simple and fast determination of the energy content it has been found advantageous to determining a stoichiometric coefficient of the hydrocarbon gas mixture. The stoichiometric coefficient of the hydrocarbon gas mixture is a coefficient that indicates a linear relation with a content of the hydrocarbon gas mixture. For example the stoichiometric coefficient of the hydrocarbon gas mixture is a coefficient of the average energy content per C atom, per H atom, per hydrocarbon compound, per volume unit, and/or per mass unit of the hydrocarbon gas mixture.

Preferably the stoichiometric coefficient is based on the content of H atoms m relative to the content of C atoms.

In an embodiment the method comprises determining a stoichiometric coefficient of the hydrocarbon gas mixture based on the content of methane relative to the content of other hydrocarbons.

In an embodiment the method comprises determining a stoichiometric coefficient of the hydrocarbon gas mixture based on the content of two or more hydrocarbons relative to the content of other hydrocarbons.

In an embodiment the method comprises determining a stoichiometric coefficient of the hydrocarbon gas mixture based on the content of at least one inert compound preferably nitrogen.

In order to find a stoichiometric coefficient for a hydrocarbon gas mixture the calibration data is prepared to comprise calculations or determinations of the energy content of a plurality of different hydrocarbon gas mixtures. The data can for example be provided as described in the conference paper “Calculation of LNG enthalpies and calorific values at different reference conditions” by Asaad Kenbar and Jurgen Rauch, ‘Metrology for LNG’, 17 & 18 Oct. 2013, De Lindenhof—Delft, The Netherlands.

In an embodiment the quality parameter comprises a combustion quality parameter, the method preferably comprising determining knock resistance of the hydrocarbon gas mixture.

In an embodiment the quality parameter comprises a mass parameter; the method preferably comprises determining mass of one or more compounds of the gas, such as of the entire hydrocarbon gas mixture.

In an embodiment the NMR reading comprises simultaneously subjecting the hydrocarbon gas mixture to a magnetic field B and a plurality of pulses of radio frequency energy E (in form of RF pulses) and receiving electromagnetic signals from the relevant isotopes such as the ¹H, the ¹³C and/or the ¹⁴N isotopes. RF pulses mean herein pulses of radio frequency energy.

It has been found that the magnetic field B need not be extremely high and that a high resolution (i.e. as low noise as possible) can be obtained even when using magnets with relatively low magnetic field and low homogenity. In an embodiment the NMR reading is performed in a magnetic field of up to about 2.5 Tesla, such as from about 0.05 Tesla to about 1.5 Tesla. Due to the relatively low magnetic field the equipment for performing the NMR reading can be kept at a surprisingly low cost while simultaneously a high resolution can be obtained.

In an embodiment the magnetic field is generated by a permanent magnet, such as a neodymium magnet. Since permanent magnets are generally not costly, this solution provides a low cost solution which for many applications may provide a sufficiently low noise result.

In an embodiment the magnetic field is generated by an electromagnet, such as a solenoid magnet or other electromagnets which are usually applied in motors, generators, transformers, loudspeakers or similar equipment. Electromagnets of high strength e.g. electromagnets that can be applied for generating a field of about 0.1 Tesla or more are often relatively expensive compared with permanent magnets. Furthermore, the electromagnet may be adjusted by adjusting the current in the coil of the electromagnet to a desired level.

In an embodiment the magnetic field is generated by a permanent magnet in combination with an electromagnet which advantageously is constructed for providing a pulsed magnetic field. By the term “pulsed magnetic field “means that the strength of the magnetic field is pulsed.

By pulsing the magnetic field even more accurate determinations can be obtained because measurements at different field strengths provide a tool for identifying noise which may accordingly be filtered off.

Advantageously the NMR reading is performed in a magnetic field with a standard deviation of the field over the sample volume of more than 30 ppm such as from about 300 ppm to 3000 ppm.

In an embodiment the method comprises transmitting narrow bandwidth RF pulses during generation of at least a part of the ¹³C data, preferably with a bandwidth of from about 10 ppm to about 200 ppm, such as from about 10 ppm to about 100 ppm, to thereby excite only a part of the ¹³C isotopes, such that the obtained ¹³C spectra reflect at least one relative amounts of two or more different carbon containing compounds, the determination is preferably performed in a magnetic field of about 0.1 to about 2 tesla and using a bandwidth of from 10 ppm to about 100 ppm, wherein the magnetic field is preferably relatively homogeneous.

The term “measuring zone” designates the zone in which the hydrocarbon gas mixture under measuring is subjected to the NMR reading e.g. determined by the zone where the hydrocarbon gas mixture is subjected to the magnetic field and the RF pulses.

In an embodiment of the invention the magnetic field in the measuring zone is preferably relatively spatially homogeneous and relatively temporally constant. However, in general it is difficult to ensure that the magnetic field in the measuring zone is entirely homogenous and further for most magnetic fields the field strength might drift or vary over time due to aging of the magnet, movement of metal objects near the magnet and/or temperature fluctuations.

Drift and variations over time can be dealt with by controlling the temperature and/or by applying a field lock and adjusting frequency such as it is generally known in the art.

Spatial inhomogeneities of the magnetic field can be alleviated by a simple calibration or alternatively or simultaneously such spatial inhomogeneities can be alleviated by shim coils such as it is also known in the art. Such shim coils may e.g. be adjusted by the computer to maximize the homogeneity of the magnetic field.

In an embodiment of the invention the method comprises performing a plurality of NMR readings at a selected magnetic field; preferably the magnetic field is kept substantially stationary during the plurality of NMR readings.

In an embodiment the method of the invention comprises regulating the temperature e.g. by maintaining the temperature at a selected value.

In an embodiment the method comprises performing the NMR reading at a fixed temperature.

In an embodiment the method of the invention comprises determining the temperature.

In an embodiment the method comprises performing a plurality of NMR readings on the hydrocarbon gas mixture. Advantageously the NMR reading of the hydrocarbon gas mixture will be performed a plurality of times in order to reduce the noise. In an embodiment the NMR reading is performed continuously in repeated measuring cycles. In an embodiment the method comprises performing a plurality of NMR readings on the same hydrocarbon gas mixture part (e.g. a sample). The NMR readings are normally performed very fast e.g. several NMR reading circles per second, such as 20 NMR readings or more, such as 50 NMR readings or more. Therefore even when performing the NMR reading on the hydrocarbon gas mixture in a flowing condition, several NMR readings may be performed on virtually the same hydrocarbon gas mixture.

In an embodiment the NMR reading comprises simultaneously subjecting the hydrocarbon gas mixture part to a magnetic field B, and an exciting RF pulse with frequencies selected to excite a nuclei spin of at least a part of the ¹H, ¹³C and/or the ¹⁴N isotope. The skilled person will be able to select suitable exciting RF pulses to provide a sufficient band width (span over a frequency range) to excite nuclei spin (spin transition) of the relevant isotopes.

In an embodiment the radio frequency pulses are in the form of adiabatic RF pulses, i.e. RF pulses that are amplitude and frequency modulated pulses.

In an embodiment of the invention the frequency range of the exciting RF pulse comprises a band width of at least about 100 KHZ.

By a few trial and error tests the desired frequency range for at specific type of determination can be found.

The actual frequencies that excite the spin of the isotope nucleus in question (¹H, ¹³C and/or ¹⁴N) depend largely on the magnetic field B. As explained above, the magnetic field may vary due to drift and due to temperature variations and it is generally preferred that the exciting RF pulses are adjusted by a field lock function in order to ensure that the NMR readings are performed using exciting RF pulses which are directed towards desired nucleus spin of the isotope.

In an embodiment the method of the invention comprises determining at least one relaxation rate of an excited isotope.

In an embodiment the ¹H NMR data comprises ¹H T1 data and/or ¹H T2 data.

In an embodiment the ¹³C NMR data comprises ¹³C T1 data and/or ¹³C T2 data.

The term relaxation describes processes by which nuclear magnetization excited to a non-equilibrium state return to the equilibrium distribution. In other words, relaxation describes how fast spins “forget” the direction in which they are oriented. Methods of measuring relaxation times T1 and T2 are well known in the art.

In an embodiment the method comprises determining at least one spin-lattice—T1 relaxation value of an excited isotope.

It is believed that T1 relaxation involves redistributing the populations of nuclear spin states in order to reach the thermal equilibrium distribution.

T1 relaxation values may be dependent on the NMR frequency applied for exciting the isotope. This should preferably be accounted for when analyzing and calibrating the T1 relaxation values obtained.

In an embodiment the method comprises determining at least one spin-spin—T2 relaxation value of an exited isotope. The T2 relaxation is also called the transverse relaxation. Generally T2 relaxation is a complex phenomenon and involves decoherence of transverse nuclear spin magnetization. T2 relaxation values are substantially not dependent on the magnetic field applied during excitation of the isotope, and for most determinations such possible variations can be ignored.

In an embodiment the method comprises subjecting the hydrocarbon gas mixture to pulsed trains of RF pulses, preferably with repetition rates of at about 1000 ms or less, such as from about 10 to about 500 ms.

The trains of RF pulses are often applied to determine the T1 and/or T2 values.

In an embodiment, the method comprises subjecting the hydrocarbon gas mixture to trains of square RF pulses, preferably with repetition rates of about 100 ms or less, such as about 10 ms or less, such as about 5 ms or less, such as about 1 ms or less.

A short square pulse of a given “carrier” frequency advantageously contains a range of frequencies centered about the carrier frequency, with the range of excitation (bandwidth/frequency spectrum) being inversely proportional to the pulse duration.

A Fourier transform of an approximately square wave contains contributions from all the frequencies in the neighborhood of the principal frequency. The restricted range of the NMR frequencies made it relatively easy to use short (millisecond to microsecond) radio frequency pulses to excite the entire NMR spectrum.

In an embodiment the NMR reading comprises simultaneously subjecting the hydrocarbon gas mixture to a magnetic field B and a plurality of RF pulses wherein the RF pulses comprise
i. an exciting RF pulse, and
ii. at least one refocusing RF pulse.

The exciting RF pulse and the refocusing pulse or pulses may for example be in the form of a train of RF pulses, e.g. pulsed pulses. The exciting RF pulse is preferably as described above and may in an embodiment be pulsed.

Useful duration and amplitude of the exciting RF pulses are well known in the art and optimization can be done by a simple trial and error.

In an embodiment the exciting RF pulse is in the form of a 90° pulse.

A 90° pulse is an RF pulse designed to rotate the net magnetization vector 90° from its initial direction in the rotating frame of reference. If the spins are initially aligned with the static magnetic field, the pulse produces transverse magnetization and free induction decay (FID).

In an embodiment the refocusing RF pulse(s) is in the form of a 180° pulse, preferably the method comprises subjecting the hydrocarbon gas mixture to a plurality of refocusing RF pulses, such as one or more trains of refocusing RF pulses.

A 90° pulse is an RF pulse designed to rotate the net magnetization vector 180° in the rotating frame of reference. Ideally, the amplitude of a 180° pulse multiplied by its duration is twice the amplitude of a 90° pulse multiplied by its duration. Each 180° pulse in the sequence (called a CPMG sequence after Carr-Purcell-Meiboom-Gill) creates an echo.

A standard technique for measuring the spin-spin relaxation time T2 utilizing CPMG sequence is as follows. As is well known after a wait time that precedes each pulse sequence, a 90-degree exciting pulse is emitted by an RF antenna, which causes the spins to start processing in the transverse plane. After a delay, an initial 180-degree pulse is emitted by the RF antenna. The initial 180-degree pulse causes the spins, which are dephasing in the transverse plane, to reverse direction and to refocus and subsequently cause an initial spin echo to appear. A second 180-degree refocusing pulse can be emitted by the RF antenna, which subsequently causes a second spin echo to appear. Thereafter, the RF antenna emits a series of 180-degree pulses separated by a short time delay. This series of 180-degree pulses repeatedly reverse the spins, causing a series of “spin echoes” to appear. The train of spin echoes is measured and processed to determine the spin-spin relaxation time T2.

In an embodiment the refocusing RF pulse(s) is/are applied with an echo-delay time after the exciting RF pulse. The echo-delay time (also called wait time TW) is preferably of about 500 μs or less, more preferably about 150 μs or less, such as in the range from about 50 μs to about 100 μs.

This method is generally called the “spin echo” method and was first described by Erwin Hahn in 1950. Further information can be found in Hahn, E. L. (1950). “Spin echoes”. Physical Review 80: 580-594, which is hereby incorporated by reference.

A typical echo-delay time is from about 10 μs to about 500 ms, preferably from about 500 μs to about 20000 μs. The echo-delay time (also called wait time TW) is the time between the last CPMG 180° pulse and the first CPMG pulse of the next experiment at the same frequency. This time is the time during which magnetic polarization or T1 recovery takes place. It is also known as polarization time.

This basic spin echo method provides very good results for obtaining T1 relaxation values by varying TW and T2. Relaxation values can also be obtained by using plurality of refocusing pulses.

The refocusing delay is also called the Echo Spacing, TE, and indicates the time identical to the time between adjacent echoes. In a CPMG sequence, the TE is the time between 180° pulses.

This method is an improvement of the spin echo method by Hahn. This method was provided by Carr and Purcell and provides an improved determination of the T2 relaxation values.

Further information about the Carr and Purcell method can be found in Carr, H. Y.; Purcell, E. M. (1954). “Effects of Diffusion on Free Precession in Nuclear Magnetic Resonance Experiments”. Physical Review 94: 630-638, which is hereby incorporated by reference.

In an embodiment the method comprises subjecting the hydrocarbon gas mixture to a polarization treatment comprising subjecting the hydrocarbon gas mixture to cross polarization to thereby determine the content of one or more of the moieties ═CH₁— moiety, —CH₂— moiety, —CH₃ moiety and/or CH₄ moiety and/or any combinations thereof, the cross polarization is preferably in the form of DEPT (Distortionless Enhancement by Polarization Transfer). The determination of the moieties can for example be applied in the determination of the energy content.

In an embodiment the method comprises enhancing signal to noise of the spectra by subjecting the hydrocarbon gas mixture to a polarization treatment during the NMR reading, the polarization treatment preferably comprises DEPT (Distortionless Enhancement by Polarization Transfer) or NOE (Nuclear Overhauser Effect) or INEPT (Insensitive Nuclei Enhanced by Polarization Transfer).

DEPT has heretofore been applied as a very useful method for determining the presence of primary, secondary and tertiary carbon atoms using 13C NMR. The DEPT experiment differentiates between CH, CH2 and CH3 groups by variation of the selection angle parameter.

The Nuclear Overhauser Effect (NOE) is the transfer of nuclear spin polarization from one nuclear spin population to another via cross-relaxation. It is a common phenomenon observed by nuclear magnetic resonance (NMR) spectroscopy. Nuclear Overhauser Effect can for example be used to determine intra- (and even inter-) molecular distances. The NOE effect is the change in population of one proton (or other nucleus) when another magnetic nucleus close in space is saturated by decoupling or by a selective 180 degree pulse.

Further information about DEPT and NOE and how to perform them can e.g. be found in New Method for NMR Signal Enhancement by Polarization Transfer, and Attached Nucleus Testing” by John Homer et al. J. Chem. Soc., Chem. Commun., 1994 and “Fundamentals of NMR” Chapter 1, by Thomas L. James, Department of Pharmaceutical Chemistry, University of California, 1998.

In an embodiment the polarization treatment comprises subjecting the hydrocarbon gas mixture to cross polarization to thereby enhance quantitative determination of relative amounts of two or more different compounds or moieties thereof, the cross polarization is preferably in the form of DEPT.

The NMR reading is advantageously performed on the hydrocarbon gas mixture in a flowing condition or in a semi flowing condition.

The phrase that the NMR reading is performed on the hydrocarbon gas mixture in a flowing condition means that the hydrocarbon gas mixture or a fraction thereof is flowing through the magnetic field during the reading. It has been observed that a high flow velocity of the hydrocarbon gas mixture in certain situations e.g. in dependence on the magnet and the generated magnet field, may result in a reduction of the pre-polarization of the gas before it reaches the measuring zone which means that it can affect the NMR readings.

The phrase that the NMR reading is performed on the hydrocarbon gas mixture in a semi flowing condition means that the hydrocarbon gas mixture is flowing through the magnetic field and temporarily stopped during at least a part of the reading.

In an embodiment the NMR reading is performed in an inline process during the transportation of the hydrocarbon gas mixture from one hydrocarbon gas mixture reservoir to another.

The NMR reading may for example be performed on the hydrocarbon gas mixture during transportation from a first fluid reservoir to a second fluid reservoir or to a point of use, such as to a storage container or to an engine

In an embodiment the NMR reading is performed on the hydrocarbon gas mixture in a flowing condition in a pipe section pumping the hydrocarbon gas mixture from a first fluid reservoir to a second fluid reservoir.

In an embodiment the NMR reading is performed in-line directly on the hydrocarbon gas mixture in a flowing condition.

The term “in-line” should herein be interpreted to mean that the NMR reading is performed directly on the hydrocarbon gas mixture source without taking a sample or samples of the hydrocarbon gas mixture and measuring on such sample(s) only. The NMR reading may e.g. be performed on the hydrocarbon gas mixture in a flowing condition as described above or it may be performed directly on the hydrocarbon gas mixture in a container.

In an embodiment the method comprises performing the NMR reading on the hydrocarbon gas mixture during its flow through a measuring zone, the NMR reading comprises subjecting the hydrocarbon gas mixture in the measuring zone to a magnetic field and a plurality of RF pulses exciting at least one type of isotopes and receiving electromagnetic signal emissioned from excited isotopes of the hydrocarbon gas mixture.

The measuring zone is advantageously in the form of a length section of a main pipe through which at least the main amount, preferably all of the hydrocarbon gas mixture is flowing.

In an embodiment the measuring zone is in the form of a length section of a by-pass pipe through which a part of the hydrocarbon gas mixture is flowing.

In an embodiment the method comprises determination of the flow velocity of the hydrocarbon gas mixture in the measuring zone.

Determination of flow velocity of gas is well known in the art and is for example performed as described in any one of the publications WO13024456, EP0496330, U.S. Pat. No. 4,816,763 and U.S. Pat. No. 3,419,795.

Preferably the determination of flow velocity is based on determining the average distance traveled by the isotopes in excited state.

Advantageously the method comprises determining a 2D flow profile of the hydrocarbon gas mixture in the measuring zone, preferably by determining the distance traveled by the isotopes in excited state as a function of the position of the isotopes in a cross-sectional 2D plane of the measuring zone.

It is well known that a partial velocity in a pipe varies in dependence on the distance to a pipe wall. The closer the wall the lower the partial velocity is. Normally when the term “velocity” is used it indicates the average flow. The term “partial velocity” is used to indicate the specific velocity in a specific distance from the pipe wall.

Further information about 2D flow profiles can be found in “Measurement of Flow Characteristics Using Nuclear Magnetic Resonance, by Marcus A. Hemminga. Biomedical Magnetic Resonance (1984)11, 157-18.

In an embodiment the method comprises performing a measurement of a pressure difference over a predefined flow path through at least a part of the measuring zone section. The length of the flow path is selected in dependence on the velocity of the hydrocarbon gas mixture or in dependent on an expected maximum velocity of the hydrocarbon gas mixture. Preferably the predefined flow path has a length of at least about 1 cm, such as from about 2 cm to about 1 m. In an embodiment the predefined flow path is from an entrance to an exit of the measuring zone of the hydrocarbon gas mixture.

In an embodiment the method comprises determining the viscosity of the hydrocarbon gas mixture based on at least the pressure difference and the flow velocity and/or the 2D flow profile.

In particular the 2D flow profile in combination with the flow velocity is useful for determining the viscosity.

The flow velocity can advantageously be applied in (e.g. for compensating for less than optimum pre-polarization at high flow velocity) determination of the quality parameter, such as a calorific parameter or alternatively the flow velocity can be applied as an additional information of the hydrocarbon gas mixture.

In an embodiment the determination of the quality parameter comprises determining at least one relationship between the ¹H NMR spectra and the ¹³C data spectra, the determination of the quality parameter preferably comprises determining at least one relationship between an average frequency response of the ¹H NMR spectra and an average frequency response of the ¹³C spectra.

In an embodiment the quality parameter is a calorific parameter in the form of energy content and the method comprises determination of the energy content of the hydrocarbon gas mixture in the form of energy flow per time-unit or energy content per mass-unit of the hydrocarbon gas mixture, preferably the energy flow per time-unit is preferably determined by determining a quality parameter comprising the energy content of an amount (mass or volume at a selected pressure and temperature) and multiplying with a determined flow of the hydrocarbon gas mixture.

In an embodiment the quality parameter comprises a calorific value, such as energy content per volume or mass unit multiplied by the flow velocity (volume or mass unit per time unit) through the measuring zone to give energy content per time unit.

In an embodiment the method comprises determining the mass flow of the gas mixture by quantitatively determining the amounts of H atoms, C atoms and N atoms based on NMR readings of ¹H and ¹³C and ¹⁴N and multiplying by their respective atomic weight and the flow velocity through the measuring zone.

In an embodiment the method comprises determination of an atomic mass per volume-unit and determination of the flow velocity of the hydrocarbon gas mixture through the NMR reader during the NMR reading and from these parameters preferably determining the total mass of the hydrocarbon gas mixture flowing through the measuring zone per time-unit.

The calibration data is advantageously based on calculations or determinations of a plurality of different hydrocarbon gas mixtures which are preferably known or at least comprise at least one known parameter.

The method of the invention advantageously comprises providing calibration data for the calibration as described above.

In an embodiment the method comprises providing calibration data of a plurality of hydrocarbon gas mixtures with different and known contents of at least one compound, preferably selected from methane, ethane, propane, butane, nitrogen and any combinations thereof.

In an embodiment the method comprises providing calibration data of a plurality of hydrocarbon gas mixtures with different and known contents of at least one of H atoms and C atoms.

In an embodiment the method comprises providing calibration data of a plurality of hydrocarbon gas mixtures with different and known contents of at least one moiety comprising a H—C bond, such as one or more of the moieties ═CH₁— moiety, —CH₂— moiety, —CH₃ moiety and/or CH₄ moiety and/or any combinations thereof.

In an embodiment the method comprises providing calibration data of hydrocarbon gas mixture mixtures with different compositions together with measured or calculated values of energy content of the respective gas mixtures.

In an embodiment the method comprises providing calibration data of hydrocarbon gas mixture mixtures with different compositions where the data is obtained at different temperature and/or pressure.

The calibration data constitutes a calibration map. The calibration map comprises the desired NMR data and optionally additionally data such as data relating to temperature(s), pH value(s) and or relative amounts of selected compounds.

The calibration map may be in the form of or comprising raw data, in the form of drawings, in form of graphs, in form of formulas or any combinations thereof.

In an embodiment, the hydrocarbon gas mixtures used for generating the calibrating map are of a similar type as the hydrocarbon gas mixture to be tested, such as is LNG, CNG or SNG. Generally it is well known in the art to calibrate NMR readings based on NMR spectra obtained on known compositions.

In an embodiment the calibration map is in the form of a pre-processed data set, where the NMR spectra obtained for a hydrocarbon gas mixture under analysis can be processed by the computer to provide the one or more quality parameters.

In an embodiment the method comprises preparing calibration data and storing the calibration data on a digital memory. The method advantageously comprises feeding the NMR data or data obtained from the NMR reading to a computer in digital communication with the digital memory and making (e.g. by using a suitable software) the computer compare and analyze the data to obtain at least one quality parameter.

The calibration map may be built up during use, for example additional data obtained by measurement on the hydrocarbon gas mixture is fed to the computer and used in the calibration of the data for later determinations.

The computer may for example be programmed to compute the data obtained using artificial intelligence or the calibration map may be applied to teach a neural network.

The invention also relates to a system suitable for determination of a quality parameter of a hydrocarbon gas mixture as described above.

The system of the invention comprises a NMR spectrometer configured for obtaining ¹H NMR data comprising a ¹H NMR spectra, a digital memory storing a calibration map comprising calibrating data for calibrating ¹H NMR data obtained by the NMR spectrometer and a computer programmed to analyze the ¹H NMR data obtained by the NMR spectrometer using the calibration map wherein the calibration data relates the ¹H NMR data to at least one quality parameter of the hydrocarbon gas mixture.

The spectrometer may be any NMR spectrometer suitable for use in performing the method of the invention. Advantageously the spectrometer is as described above and should preferably be configured to perform a NMR reading of a hydrocarbon gas mixture. Preferably the magnet(s) of the spectrometer is as described above. Advantageously the NMR spectrometer comprises one or magnets arranged to generate a substantially homogeneous magnetic field in the measuring zone of from 0.1 to 2 tesla. The calibration map may be as described above.

The calibration map may be continuously updated with new data.

The system may comprise one, two or more computers; one, two or more spectrometers and/or one, two or more calibration maps. The system may preferably be in data communication with the internet e.g. for communication with other similar systems, for sending and/or receiving data. The system may preferably comprise at least one display and/or an operating keyboard as well as any other digital equipment usually connected to digital systems, e.g. printers.

In an embodiment the system further comprises a digital memory storing a calibration map for one or more of the isotopes ¹⁴N isotope and ¹³C isotope, the map comprises calibration data for said one or more isotopes.

The system is advantageously configured to perform NMR reading on a hydrocarbon gas mixture in a flowing condition.

The NMR spectrometer comprises at least one antenna for transmitting excited RF pulses to the measuring zone and at least one antenna for receiving electromagnetic signal from the measuring zone. The transmitting antenna and the receiving antenna can be provided by the same antenna. In principle the NMR spectrometer may have as many antennas as desired. In an embodiment the NMR spectrometer comprises a plurality of antennas, such as from 2 to 20 antennas, wherein each antenna independently from the other is a transmitter, a receiver or a transceiver.

In an embodiment the system is configured to perform NMR readings on LNG at a temperature below about −100° C., such as below about −150° C., such as about −162° C.

All features of the inventions including ranges and preferred ranges can be combined in various ways within the scope of the invention, unless there are specific reasons not to combine such features.

Further scope of applicability of the present invention will become apparent from the description of examples and drawings given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF EXAMPLES AND DRAWINGS

FIG. 1 shows an example of a gas supply chain.

FIG. 2 is a schematic drawing of a system of the invention suitable for determination of a quality parameter of a hydrocarbon gas mixture of the invention.

The figures are schematic and may be simplified for clarity. Throughout, the same reference numerals are used for identical or corresponding parts.

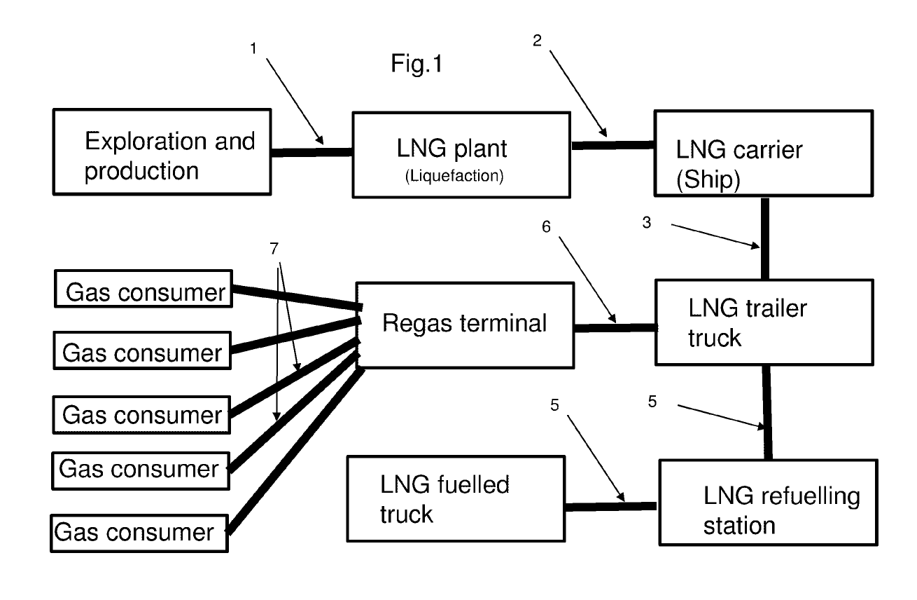

As explained above, natural gas becomes more and more important as an energy source in most of the world. FIG. 1 shows an example of a supply chain for supplying gas e.g. obtained from an off-shore plant or an on-shore plant. In the example the gas is transferred via pipeline 1 to a LNG plant where the gas is liquefied. From the LNG plant the LNG is transferred to a LNG carrier—e.g. a ship via pipeline 2. From the LNG carrier the LNG is transferred via pipeline 3 to a further carrier—here a LNG truck. The LNG trailer truck delivers LNG to a LNG refueling station via pipeline 4 from where it is delivered further to a LNG fuelled truck via pipeline 5. The LNG trailer truck also delivers LNG via pipeline 6 to a regas terminal where the gas is regassified and from where it via pipelines 7 is delivered to individual consumers.

The quality parameter of the gas can advantageously be determined using the method of the invention at one or more positions along the supply chain.

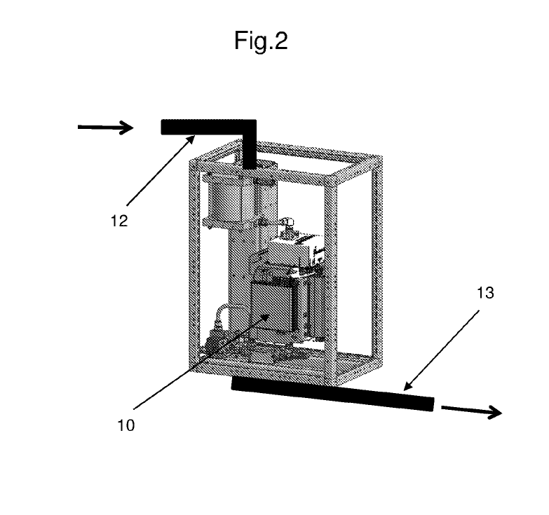

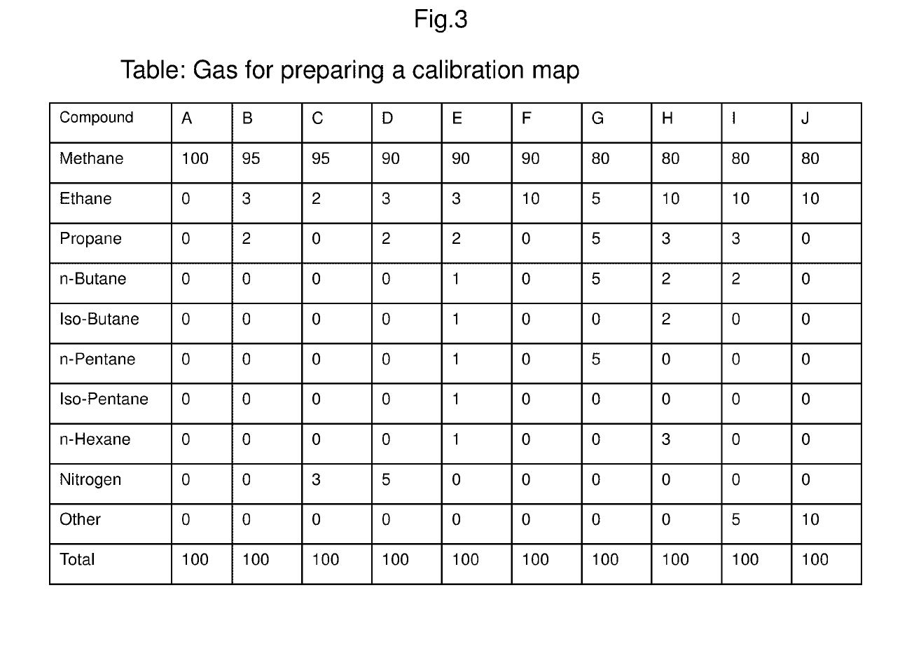

The system shown in FIG. 2 for determination of a quality parameter of a hydrocarbon gas mixture comprises a NMR spectrometer 10 configured for obtaining ¹H NMR data and preferably also ¹³C NMR data and ¹⁴N NMR data. The system further comprise a not shown digital memory storing a calibration map comprising calibrating data for calibrating the data obtained by the NMR spectrometer and a not shown computer programmed to analyze the data obtained by the NMR spectrometer using the calibration map and obtaining at least one quality parameter of the hydrocarbon gas mixture. The system comprises an in-flow pipe 12 and an out-flow pipe 13 for the hydrocarbon gas mixture such that the hydrocarbon gas mixture can be subjected to the NMR reading in a flowing or a semi flowing condition.

### Example 1

A first calibration map for obtaining a quality parameter of LNG is provided. The quality parameter includes at least an energy content.

9 LNG hydrocarbon gas mixtures with different compositions were provided as well as a pure methane gas. The compositions A-J of the hydrocarbon gas mixtures/methane are shown in FIG. 3.

The gases A-J are subjected to respective ¹H NMR readings and ¹³C NMR readings at about −162° C. and in a flow to obtain a sufficient amount of NMR data. The NMR readings may be taken at different flows for each of the sample.

The NMR data is stored in a calibration map where the data for each sample is linked to the composition of this sample and the velocity of the sample during the respective readings.

The calibration map is stored in the digital memory of the NMR system of the invention.

### Example 2

A second calibration map for obtaining a quality parameter of LNG is provided. The quality parameter includes at least an energy content.

The second calibration map is obtained in the same manner as the first calibration map of example 1 with the difference that the gases A-J are further subjected to ¹⁴N NMR readings at about −162° C. and in a flow to obtain a sufficient amount of NMR data.

### Example 3

A third calibration map for obtaining a quality parameter of LNG is provided.

The calibration map comprises a stoichiometric calculation of the energy of each component of the gases A-J based on the reaction of the hydrocarbons with oxygen into carbon dioxide and water.

The stoichiometric calculation of the energy of each of the gases A-J is stored in the calibration map where the data for each sample is linked to the composition of this sample and the calibration map is stored in the digital memory of the NMR system of the invention.

### Example 4

A fourth calibration map for obtaining a quality parameter of LNG is provided.

The fourth calibration map is obtained by obtaining at least one stoichiometric coefficient for each of the gases A-J. The at least one stoichiometric coefficient preferably comprises:
a coefficient of the average energy content per C atom;
a coefficient of the average energy content per H atom;
a coefficient of the average energy content per hydrocarbon compound;
a coefficient of the content of H atoms relative to the content of C atoms;
a coefficient of content of methane relative to the content of other hydrocarbons;
a coefficient of enthalpies of a reaction equation reacting the hydrocarbon compounds with oxygen to form carbon dioxide and water.

The one or more stoichiometric coefficients for each of the gases A-J are stored in the calibration map where the data for each sample is linked to the composition of this sample and the calibration map is stored in the digital memory of the NMR system of the invention.

### Example 5

A LNG stream with unknown composition is tested by subjecting the LNG to a NMR reading comprising at least a ¹H NMR reading and a ¹³C NMR reading and optionally a ¹⁴N NMR reading. The test is performed at about −162° C. and in a flow. The obtained data is correlated to the first calibration map (obtained in example 1) or the second calibration map (obtained in example 2) and optionally to one or both of the third and the fourth calibration maps (obtained in examples 3 and 4).

Thereby at least one quality parameter of the energy content of the LNG will be obtained.

What is claimed is:

1-48. (canceled)

49. A method for determination of a quality parameter of a hydrocarbon gas mixture, the method comprising subjecting at least a part of the hydrocarbon gas mixture to an NMR reading comprising generating a ¹H data comprising a ¹H NMR spectra and correlating the ¹H NMR data to calibration data, wherein the calibration data relates the ¹H NMR data to at least one quality parameter of the hydrocarbon gas.

50. The method of claim 49 wherein the method comprising subjecting at least a part of the hydrocarbon gas mixture to an NMR reading comprising generating a ¹⁴N data comprising a ¹⁴N NMR spectra and correlating the ¹⁴N NMR data to calibration data, wherein the calibration data relates the ¹⁴N NMR data to at least one quality parameter of the hydrocarbon gas.

51. The method of claim 49 wherein the hydrocarbon gas mixture is LNG, CNG or SNG or a mixture thereof, p the hydrocarbon gas mixture is LNG and the NMR reading is performed at a temperature below about −100° C., such as below about −150° C.

52. The method of claim 49 wherein the at least one quality parameter is selected from the content of at least one moiety comprising a H—C bond, the at least one quality parameter is selected from the content of one or more of the moieties ═CH₁— moiety, —CH₂— moiety, —CH₃ moiety and/or CH₄ moiety and/or any combinations thereof.

53. The method of claim 49 wherein the quality parameter comprises a calorific parameter such as an energy content value in the form of the energy concentration per mass unit or per volume unit at a specified pressure and temperature of the hydrocarbon gas mixture.

54. The method of claim 53 wherein the method comprises determining the energy content based on the content of C atoms and H atoms.

55. The method of claim 53 wherein the method comprises determining the energy content parameter based on reaction stoichiometry of a reaction equation of one or more of the respective hydrocarbon compounds in the hydrocarbon gas mixture, the method comprises determining the energy content parameter based on reaction stoichiometry of the reaction equation of a plurality of the respective hydrocarbon compounds in the hydrocarbon gas mixture where the reaction equation is CₙHₘ+(n+m/4)O₂->nCO₂+m/2H₂O, where n is an integer from 1 to 6 and m is 2n+2.

56. The method of claim 53 wherein the method comprises determining the energy content parameter based on Hess law on enthalpy of at least one reaction equation.

57. The method of claim 56 wherein the method comprises determining a stoichiometric coefficient of the hydrocarbon gas mixture, the stoichiometric coefficient is based on the content of H atoms m relative to the content of C atoms.

58. The method of claim 56 wherein the method comprises determining a stoichiometric coefficient of the hydrocarbon gas mixture based on the content of at least one inert compound including at least nitrogen.

59. The method of claim 49 wherein the quality parameter comprises a combustions quality parameter, the method comprising determining knock resistance of the hydrocarbon gas mixture.

60. The method of claim 49 wherein the quality parameter comprises a mass parameter, the method comprises determining mass of one or more compounds of the gas, such as of the entire hydrocarbon gas mixture.

61. The method of claim 49 wherein the method comprises performing the NMR reading on the hydrocarbon gas mixture during its flow through a measuring zone, the NMR reading comprises subjecting the hydrocarbon gas mixture in the measuring zone to a magnetic field and a plurality of RF pulses exciting at least one type of isotopes and receiving electromagnetic signal emissioned from excited isotopes of the hydrocarbon gas mixture.

62. The method of claim 61 wherein the method comprises determining a 2D flow profile of the hydrocarbon gas mixture in the measuring zone, by determining the distance traveled by the isotopes in excited state as a function of the position of the isotopes in a cross-sectional 2D plane of the measuring zone.

63. The method of claim 61 wherein the method comprises performing a measurement of a pressure difference over a predefined flow path through at least a part of the measuring zone section, wherein the predefined flow path has a length of at least about 1 cm and
wherein the method comprises determining the viscosity of the hydrocarbon gas mixture based on at least the pressure difference and the flow velocity and/or the 2D flow profile.

64. The method of claim 49 wherein the method comprises determination of a content of nitrogen atoms, the method comprises determining the concentration of nitrogen atoms and/or an amount of nitrogen atoms relative to an amount of carbon atoms and/or hydrogen atoms.

65. The method of claim 49 wherein the quality parameter comprises a calorific value such as energy content per volume or mass unit multiplied by the flow velocity (volume or mass unit per time unit) through the measuring zone to give energy content per time unit.

66. The method of claim 61 wherein the method comprises determining the mass flow of the gas mixture by quantitatively determining the amounts of H atoms, C atoms and N atoms based on NMR readings of ¹H and ¹³C and ¹⁴N and multiplying by their respective atomic weight and the flow velocity through the measuring zone.

67. The method of claim 49 wherein the NMR reading is performed in a magnetic field of up to about 2.5 Tesla, such as from about 0.05 Tesla to about 1.5 Tesla.

68. A system suitable for determination of a quality parameter of a hydrocarbon gas mixture according to the method of claim 49, the system comprises a NMR spectrometer configured for obtaining ¹H NMR data comprising a ¹H NMR spectra, a digital memory storing a calibration map comprising calibrating data for calibrating ¹H NMR data obtained by the NMR spectrometer and a computer programmed to analyze the ¹H NMR data obtained by the NMR spectrometer using the calibration map wherein the calibration data relates the ¹H NMR data to at least one quality parameter of the hydrocarbon gas mixture.
